(19) United States
(12) Patent Application Publication
(10) Pub. No.: US 2019/0110938 A1
(43) Pub. Date: Apr. 18, 2019

(54) PAD WETNESS DETECTION METHOD AND PAD WETNESS DETECTION DEVICE

(71) Applicant: Sercomm Corporation, Taipei City (TW)

(72) Inventors: Wei-Li CHIU, Taipei City (TW); Meng-Chien CHIANG, Taipei City (TW)

(21) Appl. No.: 16/029,806

(22) Filed: Jul. 9, 2018

(30) Foreign Application Priority Data
Oct. 12, 2017 (CN) 201710947576.2

(51) Int. Cl. A61F 13/42; G01N 27/12

(52) U.S. Cl. CPC A61F 13/42; A61F 2013/424; G01N 27/121

(57) ABSTRACT

A pad wetness detection method and a pad wetness detection device are provided. The pad wetness detection method includes the following steps. A plurality of initial electrical records of a medical pad are obtained via two detecting areas after a pad wetness detection device is coupled to the two detecting areas of the medical pad. A threshold value is obtained via a learning algorithm according to the initial electrical records. At least one measured electrical record of the medical pad is obtained via the two detecting areas. Whether the at least one measured electrical record reaches the threshold value is determined and a warning signal is outputted accordingly.

This application claims the benefit of People's Republic of China application Serial No. 201710947576.2, filed Oct. 12, 2017, the disclosure of which is incorporated by reference herein in its entirety.

### TECHNICAL FIELD

The disclosure relates in general to a detection method and a detection device, and more particularly to a pad wetness detection method and a pad wetness detection device.

### BACKGROUND

Along with the development of the medical technology, various medical equipment and medical aids are invented for assisting in patient care. In some cases, urinary incontinence or uncontrolled excretion may happen on the elderlies, the patients with severe illness, or the persons in the vegetative state. If the urine or the excrement is not cleaned immediately, the skin may be infected or ulcerated. Therefore, care staff must provide the medical pad for the patients and make periodic inspection to check whether the medical pad is wetted or not. However, the periodic inspection is not a suitable way because it may lead to heavy workload for the care staff.

### SUMMARY

The disclosure is directed to a pad wetness detection method and a pad wetness detection device. The pad wetness detection is performed via a learning algorithm to increase the sensitivity and the accuracy.

According to one embodiment, a pad wetness detection method is provided. The pad wetness detection method includes the following steps. A plurality of initial electrical records of the medical pad are obtained via two detecting areas after a pad wetness detection device is coupled to the two detecting areas of the medical pad. A threshold value is obtained via a learning algorithm according to the initial electrical records. At least one measured electrical record of the medical pad is obtained via the two detecting areas. Whether the at least one measured electrical record reaches the threshold value is determined and a warning signal is outputted accordingly.

According to another embodiment, a pad wetness detection device is provided. The pad wetness detection device includes a detecting unit, a learning unit, a warning unit and a processing unit. The detecting unit is configured to be coupled to two detecting areas of a medical pad. The detecting unit is used for obtaining a plurality of initial electrical records and at least one measured electrical record of the medical pad via the detecting areas. The learning unit is used for obtaining a threshold value via a learning algorithm according to the initial electrical records. The processing unit is used for determining whether any measured electrical record reaches the threshold value and for controlling the warning unit to output a warning signal accordingly.

### BRIEF DESCRIPTION OF THE DRAWINGS

FIG. 1 shows a medical pad and a pad wetness detection device according to one embodiment.

FIG. 2 shows a side view of the pad wetness detection device.

FIG. 3 shows a block diagram of the pad wetness detection device.

FIG. 4 shows a flowchart of the pad wetness detection method according to one embodiment.

FIG. 5 illustrates each step of the pad wetness detection method in FIG. 4.

FIG. 6 shows another flowchart of the pad wetness detection method according to another embodiment.

FIG. 7 illustrates each step of the pad wetness detection method in FIG. 6.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

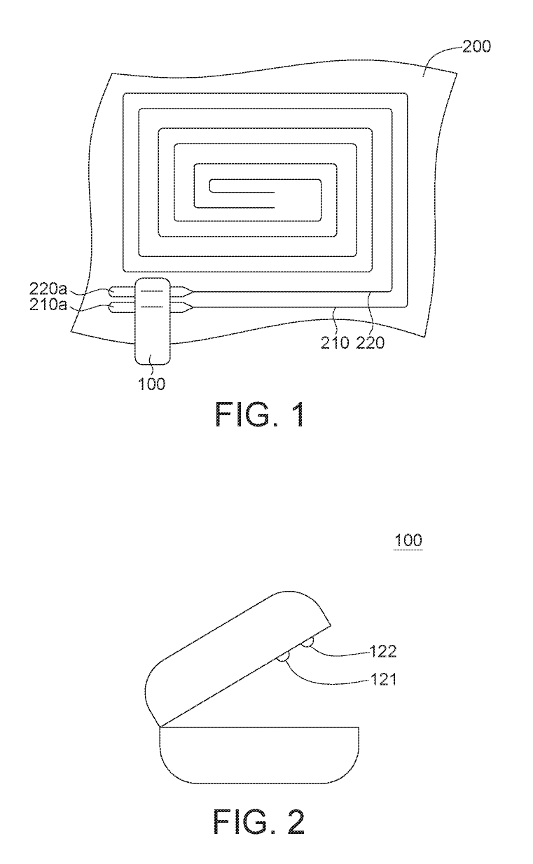

Please refer to FIG. 1, which shows a pad wetness detection device 100. A medical pad 200 is a textile pad for absorbing the liquid excreted from animal. For example, the medical pad 200 may be a bed mattress or a diaper used for human. The medical pad 200 can be used for medical application or for hygiene application. The medical pad 200 has two detecting areas 210a, 220a, The resistance between the detecting area 210a and the detecting area 220a is changed along with the wetness extent of the medical pad 200. In one embodiment, the medical pad 200 includes at least two conductive wires 210, 220 disposed therein. The conductive wires 210, 220 may be arranged in spiral at the inner layer of the medical pad 200. The ends of the conductive wires 210, 220 form the exposed detecting areas 210a, 220a. In one embodiment, the conductive wires 210, 220 can be made of carbon or conductive material. The medical pad 200 may include more than two conductive wires and more than two detecting areas corresponding thereto. In another embodiment, the medical pad 200 includes linear, strip or sheet shaped material whose resistance is changed along with the wetness extent, and the detecting areas 210a, 220a are disposed at or connected to different portions of the material, such that the resistance between the detecting area 210a and the detecting area 220a can be changed along with the wetness extent of the material.

Please refer to FIG. 2, which shows a side view of the pad wetness detection device 100. The pad wetness detection device 100 of this embodiment is a clamp. As the pad wetness detection device 100 clamps the medical pad 200 at the detecting areas 210a, 220a, two conductors 121, 122 are respectively connected to the detecting areas 210a, 220a.

When the medical pad 200 is dry, the conductive wires 210, 220 are substantially isolated. After the medical pad 200 absorbs urine, the resistance between the conductive wire 210 and the conductive wire 220 will reduce. The pad wetness detection device 100 monitors the reduction of the resistance between detecting area 210a and the detecting area 220a to determine whether the medical pad 200 is wet or not.

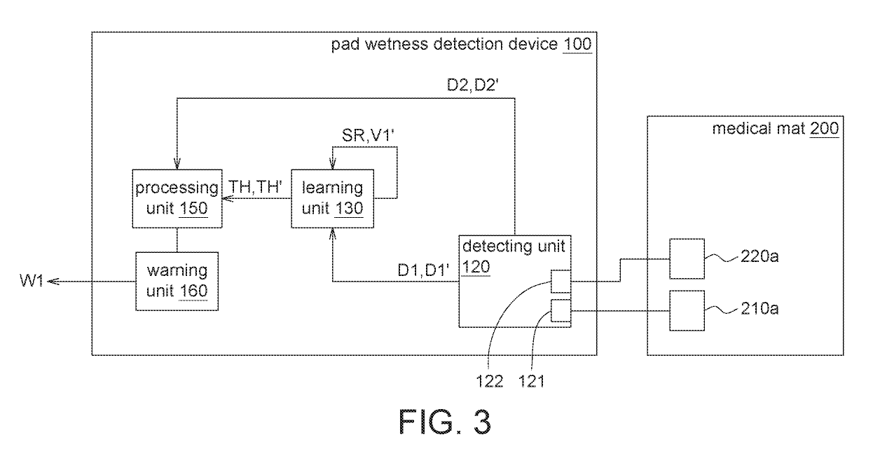

Please refer to FIG. 3, which shows a block diagram of the pad wetness detection device 100. The pad wetness detection device 100 includes a detecting unit 120, a learning unit 130, a processing unit 150 and a warning unit 160. Those components may be individual components, or those elements may be integrated into one component. Each of those components could be a part of a circuit. For example, the detecting unit 120 may include an Analog-to-Digital Converter (ADC). The learning unit 130 and the processing unit 150 may be realized by a processor. The warning unit 160 may be realized by a buzzer, a speaker, a light source or a wired/wireless transmitter.

The research staff found that if the medical pads 200 are detected by the pad wetness detection device 100 under the same threshold value, sometimes there will be detection errors, such as false positives and false negatives. The root cause of the detection errors may be the manufacturing variation of the medical pads 200, the weight of the user, the sweat from the user, the environment humidity, or the support of the medical pad, such as the bed or mattress. For increasing the accuracy of the pad wetness detection device 100, the sensitivity of the pad wetness detection device 100 can be adjusted via a learning algorithm. A flowchart is used to illustrate the operation of the pad wetness detection device 100.

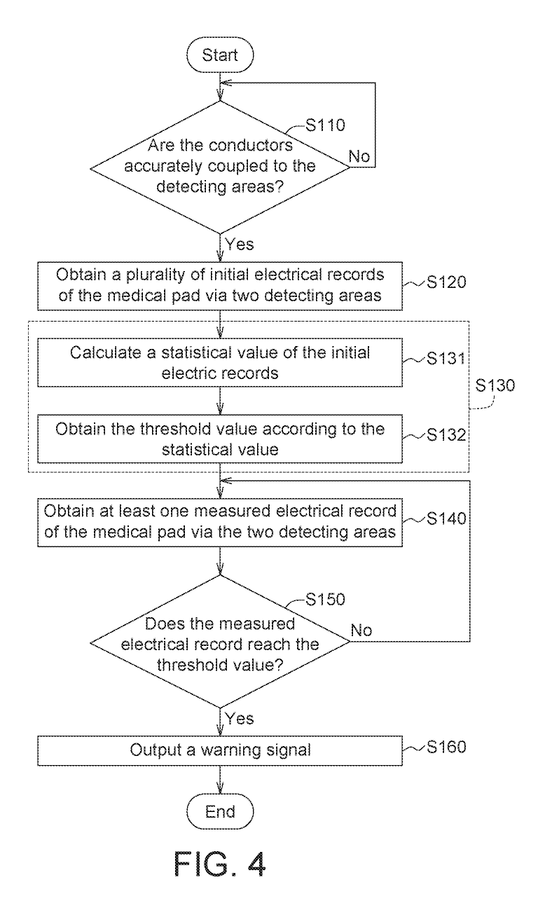

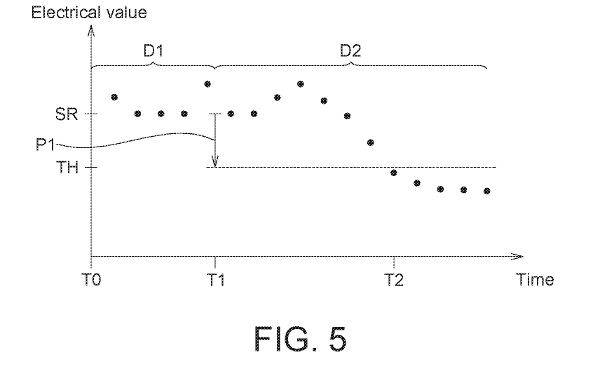

Please refer to FIGS. 4 and 5. FIG. 4 shows a flowchart of the pad wetness detection method according to one embodiment. FIG. 5 illustrates each step of the pad wetness detection method in FIG. 4. The sequence of the steps in the pad wetness detection method is not limited to the example shown in FIG. 4. Some of the steps may be omitted. Firstly, in step S110, the pad wetness detection device 100 determines whether the conductors 121, 122 are accurately coupled to the detecting area 210a and the detecting area 220a. As shown in FIG. 5, the processing unit 150 determines that the conductors 121, 122 are accurately coupled to the detecting area 210a and the detecting area 220a at time point T0.

Next, in step S120, during a predetermined period, the detecting unit 120 obtains a plurality of initial electric records D1 of the medical pad 200 via the detecting area 210a and the detecting area 220a. The predetermined period may be one minute or 30 seconds. Or, in another embodiment of the step S120, a sample number of the initial electric records D1 may be predetermined to terminate the collection of the initial electric records D1 The initial electric record D1 may be the resistance between the detecting area 210a and the detecting area 220a measured at one time point, and the resistance can be shown in digital. The initial electric records D1 reflect the influence from the weight of the user, the sweat from the user, the environment humidity, etc. According to the initial electric records D1, the sensitivity can be adjusted accordingly. As shown in FIG. 5, from the time point T0 to the time point T1, the detecting unit 120 obtains 5 initial electric records D1.

Then, in step S130, the learning unit 130 obtains a threshold value TH via a learning algorithm according to the initial electric records D1. The learning unit 130 may perform various learning algorithms. In one embodiment, the step S130 includes step S131 and step S132.

In the step S131, the learning unit 130 calculates a statistical value SR of the initial electric records D1. In the step S132, the learning unit 130 obtains the threshold value TH according to the statistical value SR. The example in the FIG. 5 shows that the statistical value SR is the mode of the initial electric records D1, and the threshold value TH is obtained by subtracting a default value P1 from the statistical value SR. In another example, the threshold value TH may be obtained by multiplying the statistical value SR and a preset value which is greater than 0 and less than 1. The statistical value may be the median or the mean.

Afterwards, in step S140, the detecting unit 120 obtains at least one measured electrical record D2 of the medical pad 200 via the detecting area 210a and the detecting area 220a. The measured electrical record D2 may be the resistance between the detecting area 210a and the detecting area 220a measured at one time point. As shown in FIG. 5, the detecting unit 120 obtains the measured electrical records D2 after the time point T1.

Next, in step S150, the processing unit 150 determines whether the measured electrical record D2 reaches the threshold value TH. If the measured electrical record D2 reaches the threshold value TH, then the process proceeds to step S160. As shown in the FIG. 5, at the time point T2, the medical pad 200 becomes wet, so the measured electrical record D2 reaches the threshold value TH. In this example, the measured electrical records D2 are positively related to the impedance between the detecting area 210a and the detecting area 220a. When the measured electrical record D2 drops and becomes lower than the threshold value TH, it is known that the impedance between the detecting area 210a and the detecting area 220a is dropped and the urinary incontinence may have occurred. In another embodiment, the measured electrical records D2 may be negatively related to the impedance between the detecting area 210a and the detecting area 220a. In this embodiment, when the measured electrical record D2 is risen and become larger than the threshold value TH, it is known that the impedance between the detecting area 210a and the detecting area 220a is dropped and the urinary incontinence may have occurred. If the threshold value TH is a lower boundary, a measured electrical record may be viewed as reach the threshold value TH if the measured electrical record is not (or no longer) higher than the threshold value TH. If the threshold value TH is an upper boundary, a measured electrical record may be viewed as reach the threshold value TH if the measured electrical record is not (or no longer) lower than the threshold value TH.

In one embodiment, the processing unit 150 makes the determination according to several measured electrical records D2 obtained in a period, to prevent from the misjudgment due to single error record. In other words, the processing unit 150 controls the process to be proceeded to the step S160 until all of M measured electrical records D2 are beneath the threshold value TH. M is an integer greater than 1.

Next, in the step S160, the warning unit 160 outputs a warning signal W1. The warning unit may be a buzzer, a speaker, a light source or a wired/wireless transmitter and the warning signal W1 may be a sound signal, a lighting signal, or a wired signal or a wireless signal transmitted to a remote device. The warning signal W1 informs the care staff of this determination.

From the time point T0 to the time point T1, the pad wetness detection device 100 is set at a learning mode. After the time point T1, the pad wetness detection device 100 is set at a detection mode. When the pad wetness detection device 100 is coupled to one new medical pad 200, the pad wetness detection device 100 enters into the learning mode first, and then the pad wetness detection device 100 enters into the detection mode. Therefore, the pad wetness detection device 100 can obtain customized threshold values TH for different medical pads, different users, and different environments via the learning algorithm.

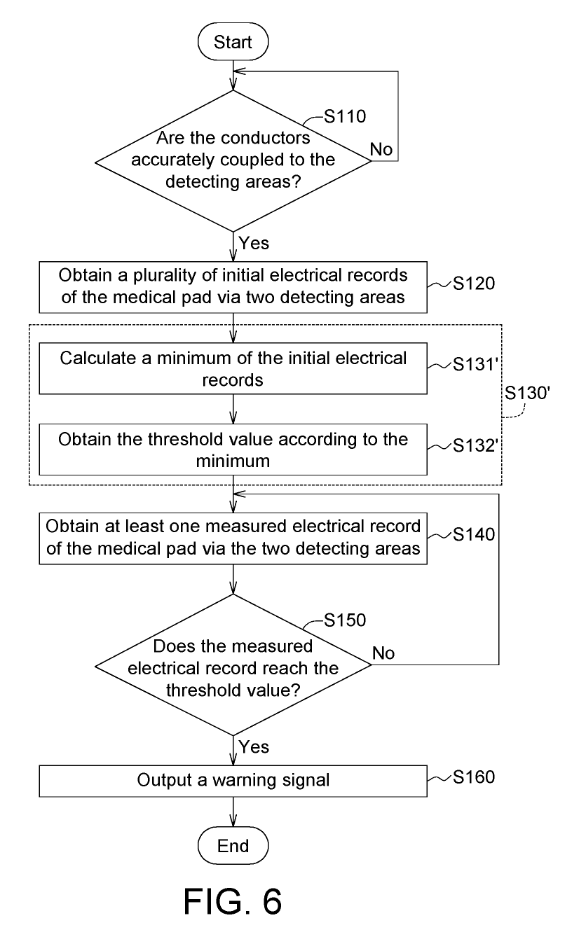

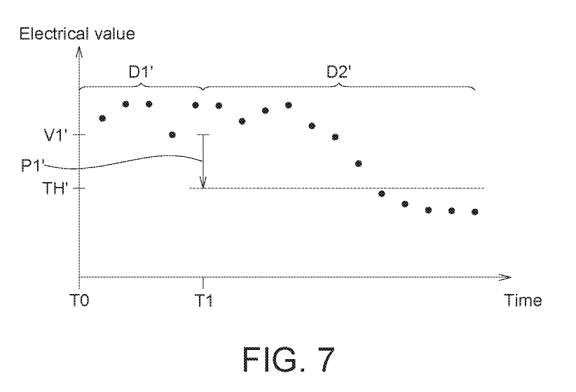

Please refer to FIGS. 6 and 7. FIG. 6 shows another flowchart of the pad wetness detection method according to another embodiment. FIG. 7 illustrates each step of the pad wetness detection method in FIG. 6. The flowchart of FIG. 6 is different from the flowchart of FIG. 5 in step S130′, and other similarities will not be repeated here. The step S130′ includes step S131′ and step S132′. In step S131′, the learning unit 130 calculates the minimum V1′ of the initial electric records D1′.

In step S132′, the learning unit 130 obtains the threshold value TH′ according to the minimum V1′. For example, as shown in FIG. 7, the threshold value TH′ may be obtained by subtracting a default value P1′ from the minimum V1′. In another embodiment, the threshold value TH′ may be obtained by multiplying the minimum V1′ and a preset value which is greater than 0 and less than 1.

That is to say, the learning unit 130 can obtain the threshold value TH′ via other learning algorithms, for different medical pads 200, different users and different environments to increase the accuracy.

It will be apparent to those skilled in the art that various modifications and variations can be made to the disclosed embodiments. It is intended that the specification and examples be considered as exemplary only, with a true scope of the disclosure being indicated by the following claims and their equivalents.

1. A pad wetness detection method, comprising:
determining whether a pad wetness detection device is coupled to two detecting areas of a medical pad;
obtaining a plurality of initial electrical records of the medical pad via the two detecting areas during a predetermined period after a time point that the pad wetness detection device is coupled to the two detecting areas of the medical pad;
setting a threshold value via a learning algorithm according to the initial electrical records which are obtained during the predetermined period;
obtaining at least one measured electrical record of the medical pad via the two detecting areas; and
determining whether the at least one measured electrical record reaches the threshold value and outputting a warning signal accordingly.

2. The pad wetness detection method according to claim 1, wherein the step of obtaining the threshold value includes:
calculating a statistical value of the initial electrical records; and
obtaining the threshold value according to the statistical value.

3. The pad wetness detection method according to claim 1, wherein the step of obtaining the threshold value includes:
calculating a minimum of the initial electrical records; and
obtaining the threshold value according to the minimum.

4. The pad wetness detection method according to claim 1, wherein in the step of outputting the warning signal, a sound signal is outputted, a lighting signal is outputted, or a wired signal or a wireless signal is transmitted to a remote device.

5. The pad wetness detection method according to claim 1, wherein the initial electrical records and the measured electrical record indicate resistances between the two detecting areas measured at different time points.

6. A pad wetness detection device, comprising:
a processing unit, used for determining whether the pad wetness detection device is coupled to two detecting areas of a medical pad;
a detecting unit, configured to be coupled to the two detecting areas of the medical pad, wherein the detecting unit is used for obtaining a plurality of initial electrical records and at least one measured electrical record of the medical pad via the detecting areas, the initial electrical records are obtained during a predetermined period after a time point that the pad wetness detection device is coupled to the two detecting areas of the medical pad;
a learning unit, used for setting a threshold value via a learning algorithm according to the initial electrical records which are obtained during the predetermined period; and
a warning unit;
wherein the processing unit is further used for determining whether the at least one measured electrical record reaches the threshold value and for controlling the warning unit to output a warning signal accordingly.

7. The pad wetness detection device according to claim 6, wherein the learning unit is used for calculating a statistical value of the initial electrical records and obtaining the threshold value according to the statistical value.

8. The pad wetness detection device according to claim 6, wherein the learning unit is used for calculating a minimum of the initial electrical records and obtaining the threshold value according to the minimum.

9. The pad wetness detection device according to claim 6, wherein the warning unit is a buzzer, a speaker, a light source or a wired/wireless transmitter.

10. The pad wetness detection device according to claim 6, wherein the initial electrical records and the measured electrical record indicate resistances between the two detecting areas measured at different time points.
